(19) United States
(12) Patent Application Publication
(10) Pub. No.: US 2013/0331777 A1
(43) Pub. Date: Dec. 12, 2013

(54) DELIVERY DEVICES AND METHODS FOR DELIVERING THERAPEUTIC AGENTS

(71) Applicants: CELL PRECISION LLC, Atlanta, GA (US); EMORY UNIVERSITY, Atlanta, GA (US)

(72) Inventors: W. Robert Taylor, Stone Mountain, GA (US); Raymond P. VIto, Atlanta, GA (US); Michael L. Wach, Alpharetta, GA (US); Jack Griffis, Decatur, GA (US)

(73) Assignees: CELL PRECISION, LLC, Altanta, GA (US); EMORY UNIVERSITY, Atlanta, GA (US)

(21) Appl. No.: 14/000,420

(22) PCT Filed: Mar. 19, 2012
(86) PCT No.: PCT/US12/29642

§ 371 Date: Aug. 20, 2013

Related U.S. Application Data
(60) Provisional application No. 61/453,691, filed on Mar. 17, 2011.

(51) Int. Cl. A61M 37/00

(52) U.S. Cl. CPC A61M 37/0069
USPC 604/59

(57) ABSTRACT

Devices and methods for delivering therapeutic agents use an inverted member to deliver a therapeutic agent with little or no shear stress. The inverted member may have a movable continuous surface, the movable continuous surface having a first section and a second section opposing the first section, the second section surrounding an inner cavity that is configured to hold the therapeutic agent, the inverting member being configured to deliver the therapeutic agent by inverting at least a portion of the second section.

### CROSS REFERENCE TO RELATED APPLICATION

This application claims priority to Provisional Application Ser. No. 61/453,691 filed Mar. 17, 2011, which is hereby incorporated by reference in its entirety.

### BACKGROUND

An emerging approach to treating disease entails encapsulating a therapeutic agent in a casing to form beads, capsules, or similar structures that a medical practitioner places in a patient adjacent diseased tissue. This approach retains the therapeutic agent at the treatment site and focuses the therapeutic agent's effect on the diseased tissue. For example, beads formed of therapeutic cells, such as stem or progenitor cells, encased in an alginate can be delivered to a strategic location of a patient's leg to treat peripheral artery disease. The casing functions as a membrane across which materials transfer to and from the therapeutic agent payload. The casing's permeability beneficially limits the sizes of the materials that can pass into or out of the bead's interior.

Conventional technologies for delivering such beads, and other systems carrying therapeutic agent payloads, generally lack sufficient sophistication for realizing full therapeutic potential. Aggressive handling resulting from these technologies, such as attempting to inject the beads with a conventional syringe, may compromise therapeutic effectiveness due to shear forces, bead-to-bead impact, and/or imprecise placement at a treatment site. Accordingly, a need exists for improved delivery methods and devices.

### SUMMARY

The disclosure relates to methods and delivery devices for delivering a therapeutic agent. These methods and delivery devices delivery a therapeutic agent with little or zero shear because the relative position of the therapeutic agent within the delivery device does not change while delivered.

In some embodiments, the delivery device may include a housing; and an inverting member having a movable continuous surface, the movable continuous surface having a first section and a second section opposing the first section, the second section surrounding an inner cavity that is configured to hold the therapeutic agent, the inverting member being configured to deliver the therapeutic agent by moving at least a portion of the second section. In some embodiments, the moving may include inverting the portion of the second section. In other embodiments, the housing may be concentrically arranged around the inverting member. In further embodiments, the housing may further include a hollow cavity configured to house the inverting member. In some embodiments, the delivery device may be configured to deliver the therapeutic agent at a distal end. In further embodiments, the housing may be a retractable sheath configured to move away from the distal end; and the inverting member may be configured to invert the portion of the second section when the retractable sheath is moved away from the distal end. In further embodiments, the delivery device may further comprise a retaining rod, the retaining rod being configured to maintain a position of the inverting member when the retractable sheath is moved.

In other embodiments, the delivery device may further comprise a retractable sleeve disposed within the housing. In further embodiments, the retractable sleeve may be configured to move away from the distal end, and the inverting member may configured to invert the portion of the second section when the retractable sheath is moved away from the distal end.

In some embodiments, the delivery device may comprise a retractable sheath; an inverting member having a movable continuous surface, the movable continuous surface having a first section and a second section opposing the first section, the second section surrounding an inner cavity that is configured to hold the therapeutic agent; and a retaining rod; wherein the retractable sheath is concentrically arranged about the retaining rod and the inverting member, the retractable sheath being configured to move away from the distal end, and the inverting member being configured to invert at least a portion of the second section when the retractable sheath is moved away from the distal end.

In further embodiments, the retractable sheath may include a first cavity and a second cavity, the retaining rod may be disposed in the first cavity and may protrude into the second cavity, and the inverting member may be disposed in the second cavity. In some embodiments, the retaining rod may be configured to maintain a position of the inverting member when the retractable sheath is moved. In other embodiments, the inverting member may be configured to be exposed when the retractable sheath is moved away from the distal end. In some embodiments, a portion of the inverting member may be attached to a portion of the retractable sheath. In further embodiments, the portion of the inverting member may be a portion of the first section adjacent to the distal end. In other embodiments, the inverting member may be configured to deliver the therapeutic agent as the portion of second section is inverted.

In some embodiments, the delivery device may comprise a housing; an inverting member having a movable continuous surface, the movable continuous surface having a first section and a second section opposing the first section, the second section surrounding an inner cavity that is configured to hold the therapeutic agent; and a retractable sleeve; wherein the retractable housing is concentrically arranged about the retractable sleeve and the inverting member, the retractable sleeve being configured to move with respect to the housing away from the distal end, and the inverting member being configured to invert at least a portion of the second section when the retractable sleeve is moved away from the distal end.

In some embodiments, the housing may include a first cavity and a second cavity, the housing including a plurality of retaining prongs between the first cavity and the second cavity; the inverting member may be disposed in the second cavity; and the retaining prongs may be configured to hold the inverting member in the second cavity. In further embodiments, the inverting member may be configured to deliver the therapeutic agent as the portion of second section is inverted. In other embodiments, the delivery device may be a part of a delivery system, wherein the proximal end of the delivery device is connected to the delivery system. In some embodiments, the housing may include four retaining prongs disposed around the housing at 90°. In some embodiments, the housing may include a retaining edge configured to maintain a position of the inverting member during use

In further embodiments, the disclosure relates to a method of delivering a therapeutic agent. The method may comprise pulling a retractable sheath that engages an inverting member while holding a retaining rod, the inverting member having a movable continuous surface, the movable continuous surface having a first section and a second section opposing the first section, the second section surrounding an inner cavity that is configured to hold the therapeutic agent; the pulling causing at least a portion of the second section to invert and become exposed; and delivering the therapeutic agent as the portion of the second section inverts.

In other embodiments, the method may comprise pulling a retractable sleeve that engages an inverting member while holding a housing, the inverting member having a movable continuous surface, the movable continuous surface having a first section and a second section opposing the first section, the second section surrounding an inner cavity that is configured to hold the therapeutic agent; the pulling causing at least a portion of the second section to invert and face the housing; and delivering the therapeutic agent as the portion of the second section inverts. In some embodiments, the method may comprise positioning an inverting member holding a therapeutic agent adjacent to a treatment site; causing a section of the inverting member to invert; and releasing the therapeutic agent from the inverting member.

Additional advantages of the disclosure will be set forth in part in the description which follows, and in part will be obvious from the description, or may be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### BRIEF DESCRIPTION OF THE DRAWINGS

The disclosure can be better understood with the reference to the following drawings and description. The components in the figures are not necessarily to scale, emphasis being placed upon illustrating the principles of the disclosure.

FIG. 1 is a cross-sectional view of a delivery device according to an embodiment; and

FIG. 2 is a cross-sectional view of a delivery device according to another embodiment.

### DESCRIPTION OF THE EMBODIMENTS

The following description, numerous specific details are set forth such as examples of specific components, devices, methods, etc., in order to provide a thorough understanding of embodiments of the disclosure. It will be apparent, however, to one skilled in the art that these specific details need not be employed to practice embodiments of the disclosure. In other instances, well-known materials or methods have not been described in detail in order to avoid unnecessarily obscuring embodiments of the disclosure. While the disclosure is susceptible to various modifications and alternative forms, specific embodiments thereof are shown by way of example in the drawings and will herein be described in detail. It should be understood, however, that there is no intent to limit the disclosure to the particular forms disclosed, but on the contrary, the disclosure is to cover all modifications, equivalents, and alternatives falling within the spirit and scope of the disclosure.

It will be understood that the delivery devices according to the embodiments may be implanted into a patient with use of a delivery system. The delivery devices may be a part of the delivery system. The proximal end (not shown in detail) of the disclosed delivery devices may be attached or connected to the delivery system. The disclosed delivery devices may be either part of the main body of the delivery system or may be a detachable component, such as a cartridge. The disclosed delivery devices may be attached to the distal end of a delivery system for insertion into a patient. The delivery system may be any known delivery system, including, but not limited to, a trocar or a cannula, or a flexible catheter. It will also be understood that a clinician may use a delivery system to control the operation of the disclosed delivery devices. It will be further understood that although the operations of the disclosed delivery devices are discussed with respect to the actions of a clinician, delivery systems may be configured to perform these actions.

The delivery systems and the disclosed devices may be adjusted and sized for the anatomy and the direction of the desired point of insertion. For example, the delivery systems and devices may be sized for lengthwise insertion into a vascular lumen. The disclosed delivery devices may be further adjusted according to the requirements of the therapeutic agent(s) to be delivered to the patient. The requirements may include, but not limited to, the size and the dosage amount of the therapeutic agent(s).

The delivery devices and systems according to embodiments may be configured to deliver a therapeutic agent(s). The therapeutic agent(s) may include any one or more substance, compound, composition, formulation, and/or agent capable of exerting an effect, such as an therapeutic, prophylactic or diagnostic effect, on a patient. The therapeutic agent(s) may be embedded or dispersed in alginate strings or filaments, encapsulated, suspended in a highly viscous solution, suspended in a high-volume fraction solution, as well as provided in any known vehicle to deliver a therapeutic agent. Such strings or filaments may be end loaded into the distal end of the delivery devices and systems according to embodiments, without necessarily feeding from the proximal end to the distal end. Suitable techniques for making therapeutic agent(s), for example, making strings and filaments comprising cells and alginate, are known in the art and may be readily created by those of ordinary skill having benefit of the present teaching. See, for example, “Grafting microcapsules of genetically modified cells: Therapeutic potential in spinal cord injury;” M. Wheatley, N. Dhoot, S. Kanakasabai and I. Fischer; Drexel University, Philadelphia, USA; XVth International Workshop on Bioencapsulation, Vienna, Au.; Sep. 6-8, 2007; S7-2, pages 1-4.

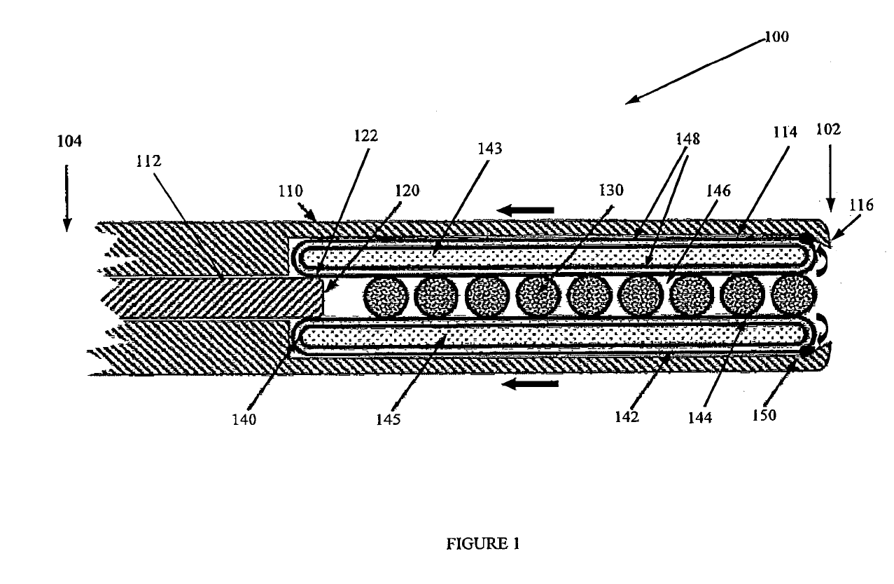

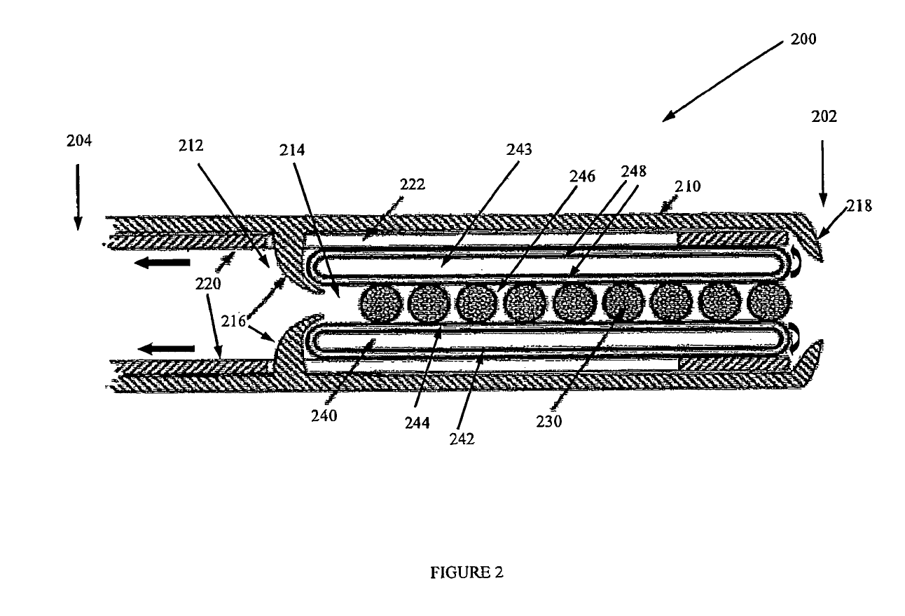

FIGS. 1 and 2 show cross-sections of delivery devices according to embodiments. FIG. 1 shows one example of a delivery device according to some embodiments. A delivery device 100 may include a retractable sheath 110, a retaining rod 120, and an inverting member 140, as shown in FIG. 1.

In some embodiments, the housing or retractable sheath 110 (hereinafter referred to as “retractable sheath”) may be a tube. In some embodiments, the retractable sheath 110 may be an outer cannula. The retractable sheath 110 may be concentrically arranged about the retaining rod 120 and the inverting member 140. In further embodiments, the retractable sheath 110 may have at least two inner cavities. In some embodiments, the retractable sheath 110 may have two inner cavities having different diameters. As shown in FIG. 1, the retractable sheath 110 may have a first inner cavity 112 and a second inner cavity 114. The first inner cavity 112 may be configured to house the retaining rod 120. In some embodiments, the first inner cavity 112 may have a diameter and length that corresponds to the diameter and length of the retaining rod 120, respectively. The second inner cavity 114 may be configured to house the inverting member 140. In some embodiments, the first inner cavity 112 may have a diameter and length that corresponds to the diameter and length of the inverting member 140, respectively.

The retractable sheath 110 may also have a tapered edge 116 on the distal end 102. The tapered edge 116 may be along the entire distal end 102. The tapered edge 116 may be a flat or angled surface.

The retaining rod 120 may be a straight piece of material. The retaining rod 120 may be any shape, including but not limited to, round or square. The diameter of the retaining rod 120 may correspond to the diameter of the inner area of the inverting member 140 (discussed in more detail below). The retaining rod 120 may be disposed within the first cavity 112 and a portion of the second cavity 114. The retaining rod 120 may protrude into an inner cavity 146 of the inverting member 140. In some embodiments, the retaining rod 120 may be mated with the inner cavity 146 of the inverting member 140. In other embodiments, the retaining rod 120 may be fixedly disposed to the inverting member 140. The retaining rod may be fixedly disposed within the inner cavity 146 by a fastener 122. The fastener 122 may be any known fastener, such as a biocompatible adhesive.

In some embodiments, the retractable sheath 110 and the retaining rod 120 may be made of a biocompatible material, such as a hypodermic metal, including, but not limited to stainless steel. The retractable sheath 110 and the retaining rod 120 may be made of the same material or a different material. The length and the diameter of the retractable sheath 110 and the retaining rod 120 may be based on the size (e.g., diameter) of the therapeutic agent to be delivered.

In some embodiments, the inverting member 140 may have a hollow-walled tubular structure. The inverting member 140 may have a movable continuous surface 148 that forms an outer surface as well as an inner surface that surrounds an inner cavity 146 and is capable of moving so that the inner surface becomes the outer surface. The inner cavity 146 may be along the length of the inverting member 140.

In some embodiments, the movable continuous surface 148 of the inverting member 140 may have a first section 142 and a second section 144 that opposes the first section 142. When the inverting member 140 is in a static position, the first section 142 of the inverting member 140 is the surface(s) of the inverting member closest to and face the retractable sheath 110, and the second section 144 of the inverting member 140 is the surface(s) that surround the inner cavity 146.

The movable continuous surface 148 of the inverting member 140 is configured to rotate so that the second section 144 inverts. In some embodiments, when a force is applied to the inverting member 140 in the direction from the distal end to the proximal end, the first section 142 moves toward the proximal end causing the second section 144 to also move and invert.

The movable continuous surface 148 of the inverting member 140 may be made of a low friction material, such as a biocompatible elastomeric material, including, but not limited to, polyurethane, polyethylene, silicone rubber, a polymer film, or other low durometer.

The inverting member 140 may include a void 143 between the first section 142 and the second section 144. The void 143 may be filled with a low viscosity biocompatible material 145. In some embodiments, the material 145 may be silicone oil or a biocompatible lubricant, including, but not limited to, hyaluronic acid, lubricin, and natural lubricants produced by the body and present in the synovial fluid or in the joints.

In some embodiments, the inner cavity 146 may be configured to house (hold) a therapeutic agent 130 to be delivered. The dimensions of the inner cavity 146 may be configured according to the amount and the type of therapeutic agent 130 to be housed and delivered. In some embodiments, the therapeutic agent 130 may be encapsulated. Examples of encapsulation may include cells, hollow like porous spheres made of alginate, or drugs contained in nanotubes. The therapeutic agent 130 is not limited to encapsulation within spheres as shown in FIG. 1, and may also include any known vehicle to deliver a therapeutic agent. For example, in other embodiments, the therapeutic agent 130 may be therapeutic substances suspended in a highly viscous solution, suspended in a high-volume-fraction solution, embedded in strings or filaments, as well as suspended or embedded in other packages.

As shown in FIG. 1, in some embodiments, the inverting member 140 may be fixedly attached to the retractable sheath 110. In some embodiments, a portion of the first section 142 closest to the tapered edge 116 may be fixedly attached to the retractable sheath 110, as shown in FIG. 1. In other embodiments, a different portion of the first section 142 may be attached to the retractable sheath 110. The first section 142 of the inverting member 140 may be attached to the retractable sheath 110 using a fastener 150. The fastener 150 may be any known fastener. In some embodiments, the fastener 150 may be a biocompatible adhesive.

In operation, the clinician positions the tapered edge 116 (i.e., the distal end 102) of the delivery device 100 adjacent to a treatment site intended to be treated, such as a diseased tissue. Working from the proximal end 104 of the delivery device 100 (not shown) protruding from the patient, the clinician holds the retaining rod 120 in a fixed position and slides the retractable sheath 110 back so that the retractable sheath 110 is moved toward the proximal end 104 of the delivery device 100. The retractable sheath 110 is thus retracted relative to the retaining rod 120. While the retractable sheath 110 is being moved towards the proximal end 104 of the delivery device 100, the inverting member 140 also moves so as to become inverted. The first section 142 moves along with the retractable sheath 110 (due to fastener 150) thereby causing the second section 144 to also move and inert. The movement of the retractable sheath 110 also causes portions of the inverting member 140 to become exposed. At least a portion of the second section 144 becomes exposed because the movement causes the second section 144 to become the exterior surface as it rotates past the distal end. The inversion or rotation of the second section 144 at the distal end 102 causes the delivery or release of the therapeutic agent 130 from the delivery device 100 to the treatment site.

In other embodiments, the delivery device may be adapted to eject the therapeutic agent from its distal end. FIG. 2 shows a delivery device according to these embodiments.

A delivery device 200 may include a housing 210 that holds a retractable sleeve 220 and an inverting member 240. The delivery device 200 may have a distal end 202 and a proximal end 204 (not shown).

In some embodiments, the housing 210 may be a tube. In other embodiments, the housing 210 may be an outer cannula and the retractable sleeve 220 may be an inner cannula. In some embodiments, the housing 210 may be concentrically arranged about the retractable sleeve 220 and the inverting member 240.

In further embodiments, the housing 210 may have at least two inner cavities. In some embodiments, the housing 210 may have two inner cavities of the same inner diameter. In other embodiments, the housing 210 may have different sized cavities.

As shown in FIG. 2, the housing 210 may have a first inner cavity 212 and a second inner cavity 214. In some embodiments, the second inner cavity 214 may be configured to house the inverting member 240 and a portion of the retractable sleeve 220. In some embodiments, the second inner cavity 214 may have a length that corresponds to at least the length of the inverting member 240.

In some embodiments, the housing 210 may further include a plurality of retaining prongs 216 that maintain the position of the inverting member 240 during operation of the delivery device 200. There may be any number and configuration of the retaining prongs 216. The number and configuration, such as angle, of the retaining prongs 216 may be based on the shape and size of the inverting member 240. In further embodiments, there may be four retaining prongs disposed around the housing at 90°.

In some embodiments, the housing 210 may also include a retaining edge 218. The retaining edge 218 may also maintain the position of the inverting member 240 during use. The number and configuration, such as angle, of the retaining edge may be based on the shape and size of the inverting member 240.

In some embodiments, the retractable sleeve 220 may include slots 222. In some embodiments, the retractable sleeve 220 may include four slots at 90°.

The housing 210 and retractable sleeve 220 may be made of a biocompatible material, such as a hypodermic metal including, but not limited to stainless steel. The housing 210 and retractable sleeve 220 may be made of the same material or a different material.

In some embodiments, the inverting member 240 may have a hollow-walled tubular structure. The inverting member 240 may be the same as the inverting member 140 discussed above with respect to FIG. 1. The inverting member 240 may have a movable continuous surface 248 that forms an outer surface as well as an inner surface that surrounds an inner cavity 246 and is capable of moving so that the inner surface becomes the outer surface. The inner cavity 246 may be along the length of the inverting member 240.

In some embodiments, like the inverting member 140, the movable continuous surface 248 of the inverting member 240 may have a first section 242 and a second section 244 that opposes the first section 242. When the inverting member 240 is in a static position, the first section 242 of the inverting member 240 is the surface(s) closest to and face the housing 210, and the second section 244 of the inverting member 240 is the surface(s) that surround the inner cavity 246.

The movable continuous surface 248 of the inverting member 240 may be configured to rotate so that the second section 244 inverts. The inverting member 240 may be made of a low friction material, such as a biocompatible elastomeric material, including, but not limited to, a thermoplastic material, polyurethane, polyethylene, silicone rubber, a polymer film, or other low durometer.

Like the inverting member 140 in FIG. 1, the inverting member 240 may include a void 243 between the first section 242 and the second section 244. The void 243 may be filled with a low viscosity biocompatible material like the material 143. In some embodiments, the material may be a silicone oil or a biocompatible lubricant, including, but not limited to, hyaluronic acid, lubricin, and natural lubricants produced by the body and present in the synovial fluid or in the joints.

In some embodiments, the inner cavity 246 may be configured to house a therapeutic agent 230 to be delivered. The dimensions of the inner cavity 246 may be configured according to the amounts and the kind of therapeutic agent 230 to be housed and delivered. In some embodiments, the therapeutic agent 230 may be encapsulated. Examples of encapsulation may include cells, hollow like porous spheres made of alginate, or drugs contained in nanotubes. The therapeutic agent 230 is not limited to encapsulation within spheres as shown in FIG. 2, and may also include any known vehicle to deliver a therapeutic agent. For example, in other embodiments, the therapeutic agent 230 may be therapeutic substances suspended in a highly viscous solution, suspended in a high-volume-fraction solution, embedded in strings or filaments, as well as suspended or embedded in other packages.

In operation, the clinician positions the retaining edge 218 (i.e., the distal end 202) of the delivery device 200 adjacent to a treatment site intended for treatment, such as a diseased tissue. Working from the proximal end 204 of the delivery device 200 protruding from the patient, the clinician holds the proximal end of the housing 210 (not shown in detail) in a fixed position and slides the retractable sleeve 220 back so that the retractable sleeve 220 is moved toward the proximal end 204 of the delivery device 200. The retractable sleeve 220 is thus retracted relative to the housing 210. While the retractable sleeve 220 is being moved towards the proximal end 204 of the delivery device 200, the inverting member 240 is being rotated so at least a portion of the second section 244 becomes inverted within the second cavity 214. Moving the retractable sleeve 220 backwards towards the proximal end 204 causes friction against the first section 242 causing the first section 242 to also move towards the proximal end 204, which in turn causes at least a portion of second section 242 to invert. The first section 242 moves closer to the proximal end 204. This movement in turn causes at least a portion of the second section 244 to rotate and invert. Portion(s) of the second section 244 that invert thereby face or are closest to the housing 210. Rotating or inverting the second section 244 also causes the delivery or release of the therapeutic agent 230 from the delivery device 200 to the treatment site.

The advantages of the disclosed delivery devices are that the relative position of the therapeutic agent to the inside [contacting] surface of the delivery devices does not change. The inverting member of the disclosed devices has a structure similar to a “water snake” or “water wigglie” toy. The section of the inverting member that surrounds the inner cavity maintains constant contact with the therapeutic agent as the surface of the inverting member moves. As such, highly viscous solutions or conglomerations of therapeutic “beads” may be deployed externally to the delivery system with minimal effort for the clinician or minimized discomfort for the patient. In addition, the resulting delivery may have a low or zero “shear” event for the agent, minimizing negative affects to therapeutic efficacy.

In some embodiments, the delivery device may be sterilized. In further embodiments, the delivery device may be a single, use device. In further embodiments, the delivery device may be disposable. In some embodiments, the delivery device may be preloaded with the therapeutic agent. In other embodiments, the agent may be loaded into the delivery device via a cartridge.

All references cited herein are hereby incorporated by reference in their entirety.

While the disclosure has been described in detail with reference to exemplary embodiments, those skilled in the art will appreciate that various modifications and substitutions can be made thereto without departing from the spirit and scope of the disclosure as set forth in the appended claims. For example, elements and/or features of different exemplary embodiments may be combined with each other and/or substituted for each other within the scope of this disclosure and appended claims.

What is claimed is:

1. A delivery device for delivering a therapeutic agent, comprising:
a housing; and
an inverting member having a movable continuous surface, the movable continuous surface having a first section and a second section opposing the first section, the second section surrounding an inner cavity that is configured to hold the therapeutic agent, the inverting member being configured to deliver the therapeutic agent by moving at least a portion of the second section.

2. The delivery device according to claim 1, wherein the housing is concentrically arranged around the inverting member.

3. The delivery device according to claim 1, wherein the housing further includes a hollow cavity configured to house the inverting member.

4. The delivery device according to claim 1, wherein:
the delivery device is configured to deliver the therapeutic agent at a distal end, the delivery device further comprising:
the housing is a retractable sheath configured to move away from the distal end; and
the inverting member is configured to invert the portion of the second section when the retractable sheath is moved away from the distal end.

5. The delivery device according to claim 4, further comprising:
a retaining rod, the retaining rod being configured to maintain a position of the inverting member when the retractable sheath is moved.

6. The delivery device according to claim 1, further comprising:
a retractable sleeve disposed within the housing,
wherein the retractable sleeve is configured to move away from the distal end, and
wherein the inverting member is configured to invert the portion of the second section when the retractable sheath is moved away from the distal end.

7. A delivery device for delivering a therapeutic agent at a distal end, comprising:
a retractable sheath;
an inverting member having a movable continuous surface, the movable continuous surface having a first section and a second section opposing the first section, the second section surrounding an inner cavity that is configured to hold the therapeutic agent; and
a retaining rod;
wherein the retractable sheath is concentrically arranged about the retaining rod and the inverting member, the retractable sheath being configured to move away from the distal end, and the inverting member being configured to move at least a portion of the second section when the retractable sheath is moved away from the distal end.

8. The delivery device according to claim 7, wherein:
the retractable sheath includes a first cavity and a second cavity;
the retaining rod is disposed in the first cavity and protrudes into the second cavity; and
the inverting member is disposed in the second cavity.

9. The delivery device according to claim 8, wherein the retaining rod is configured to maintain a position of the inverting member when the retractable sheath is moved.

10. The delivery device according to claim 8, wherein the inverting member is configured to be exposed when the retractable sheath is moved away from the distal end.

11. The delivery device according to claim 8, wherein a portion of the inverting member is attached to the portion of the retractable sheath.

12. The delivery device according to claim 11, wherein the portion of the inverting member is a portion of the first section adjacent to the distal end.

13. The delivery device according to claim 8, wherein the portion of the second section is inverted when the retractable sheath is moved away from the distal end.

14. The delivery device according to claim 13, wherein the inverting member is configured to deliver the therapeutic agent as the portion of second section is inverted when the retractable sheath is moved away from the distal end.

15. A delivery device for delivering a therapeutic agent at a distal end, comprising:
a housing;
an inverting member having a movable continuous surface, the movable continuous surface having a first section and a second section opposing the first section, the second section surrounding an inner cavity that is configured to hold the therapeutic agent; and
a retractable sleeve;
wherein the housing is concentrically arranged about the retractable sleeve and the inverting member, the retractable sleeve being configured to move with respect to the housing away from the distal end, and the inverting member being configured to move at least a portion of the second section when the retractable sleeve is moved away from the distal end.

16. The delivery device according to claim 15, wherein:
the housing includes a first cavity and a second cavity, the housing including a plurality of retaining prongs between the first cavity and the second cavity;
the inverting member is disposed in the second cavity; and
the retaining prongs being configured to hold the inverting member in the second cavity.

17. The delivery device according to claim 16, wherein the delivery device includes four retaining prongs disposed around the housing at 90°.

18. The delivery device according to claim 15, wherein the portion of the second section is inverted when the retractable sleeve is moved away from the distal end.

19. The delivery device according to claim 18, wherein the inverting member is configured to deliver the therapeutic agent as the portion of second section is inverted.

20. The delivery device according to claim 15, wherein the housing includes a retaining edge configured to maintain a position of the inverting member during use.
